(19) United States
(12) Patent Application Publication
(10) Pub. No.: US 2008/0200764 A1
(43) Pub. Date: Aug. 21, 2008

(54) ENDOSCOPE SYSTEM

(75) Inventor: Shinsuke OKADA, Saitama (JP)

Correspondence Address: GREENBLUM & BERNSTEIN, P.L.C., 1950 ROLAND CLARKE PLACE, RESTON, VA, 20191, US

(73) Assignee: PENTAX CORPORATION, Tokyo (JP)

(21) Appl. No.: 12/018,931

(22) Filed: Jan. 24, 2008

(30) Foreign Application Priority Data
Feb. 15, 2007 (JP) 2007-035051

(51) Int. Cl. A61B 1/12

(52) U.S. Cl. 600/157

(57) ABSTRACT

An endoscope system, including an endoscope having a first optical system to obtain an image of an observation object in predetermined magnifying power and a first cleaning system to discharge fluid toward a front end surface of the first optical system to clean the front end surface, and a processor to process the image obtained through the endoscope, having a light source to illuminate the observation object and a reservoir to store the fluid for cleaning, is provided. A dischargeable portion of the first cleaning system is protruded forward from the front end surface of the first optical system when the fluid is discharged from the dischargeable portion and retracted rearward when the image of the observation object is obtained by the first optical system.

### BACKGROUND OF THE INVENTION

The present invention relates to an endoscope capable of being inserted into a body cavity to capture images of body tissue and an endoscope system having the endoscope.

Recently, a so-called confocal endomicroscope system having a confocal optical system, which is capable of capturing images of an object in a body cavity in larger magnification and higher resolution, has been introduced. In such a confocal endomicroscope system, images in the larger magnification and the higher resolution are reproduced according to electric signals, which are generated based on lights from the object being solely received through a pinhole provided in a position conjugating with a focal point of the endoscope system. An example of such a confocal endomicroscope system is disclosed in Japanese Patent Provisional Publication No. 2005-640.

In the confocal endomicroscope system disclosed in the above-referenced publication, a tip end surface of the confocal optical system is brought to be in contact with the body tissue when images of the object are generated. Therefore, the tip end surface can be easily fouled by blots of body fluid, for example, and such blots can deteriorate images being generated. However, in conventional confocal endomicroscope systems, no specific or effective configuration to clean the tip end surface of the confocal endomicroscope system has been suggested.

### SUMMARY OF THE INVENTION

In view of the above, the present invention is advantageous in that an endoscope system, wherein the tip end surface of an optical system, specifically a confocal optical system, can be effectively cleaned, while image capturing and observation of the image through the optical system is secured, is provided.

According to an aspect of the present invention, there is provided an endoscope system. The endoscope system includes an endoscope having a first optical system to obtain an image of an observation object in predetermined magnifying power and a first cleaning system to discharge fluid toward a front end surface of the first optical system to clean the front end surface, and a processor to process the image obtained through the endoscope, having a light source to illuminate the observation object and a reservoir to store the fluid for cleaning. A dischargeable portion of the first cleaning system is protruded forward from the front end surface of the first optical system when the fluid is discharged from the dischargeable portion and retracted rearward when the image of the observation object is obtained by the first optical system.

Optionally, the first optical system may be a confocal optical system, which is placed to be in direct contact with the observation object, capable of emitting illuminating light to the observation object and extracting light from the observation object, which is in a predetermined position with respect to the first optical system.

Optionally, the dischargeable portion of the first cleaning system may be retracted rearward to align on a plane on which the front end surface is positioned when the image of the observation object is obtained by the first optical system.

Optionally, the dischargeable portion of the first cleaning system may be retracted rearward from a plane on which the front end surface is positioned when the image of the observation object is obtained by the first optical system.

Optionally, the dischargeable portion of the first cleaning system may be protruded forward from the front end surface of the first optical system by pressure of the fluid to be discharged from the dischargeable portion.

Optionally, the dischargeable portion of the first cleaning system may be formed by at least one deformable resilient member. The dischargeable portion may be protruded forward when the resilient member is deformed by the pressure of the fluid.

Optionally, the resilient member may be provided in a position of the dischargeable portion to form a dischargeable opening when the fluid is fed in the dischargeable portion and the resilient member is deformed by the pressure of the fluid.

Optionally, the dischargeable opening may be closed when the fluid is unfed in the dischargeable portion.

Optionally, the dischargeable portion of the first cleaning system may be provided with a resilient spring. The resilient spring may be extended when the fluid is fed in the dischargeable opening so that the dischargeable portion is protruded forward from the front end surface of the first optical system by the pressure of the fluid and compressed when the fluid is unfed in the dischargeable portion so that the dischargeable portion is retracted.

Optionally, the dischargeable portion of the first cleaning system may be provided with at least one compressible member. The dischargeable portion of the first cleaning system may be retracted rearward to align on the plane on which the front end surface of the first optical system is positioned when the front end surface is placed to be in direct contact with the observation object for confocal observation and the resilient member is compressed by pressure to place the front end surface of the first optical system in direct contact with the observation object.

Optionally, the dischargeable portion of the first cleaning system may be provided with at least one compressible member. The dischargeable portion of the first cleaning system may be retracted rearward from the plane on which the front end surface is positioned when the front end surface is placed to be in direct contact with the observation object for confocal observation and the resilient member is compressed by pressure to place the front end surface of the first optical system in direct contact with the observation object.

Optionally, the endoscope system may include a second optical system to obtain the image of the observation object in different magnifying power from the magnifying power of the first optical system, and a second cleaning system to discharge the fluid toward a front end surface of the second optical system to clean the front end surface.

Optionally, the magnifying power of the first optical system may be greater than the magnifying power of the second optical system. The front end surface of the first optical system may be protruded forward further than the front end surface of the second optical system.

Optionally, the first cleaning system and the second cleaning system may be fed with the fluid being stored in the reservoir of the processor. The endoscope system may be provided with a switching system to switch flow of the fluid to be fed to one of the first cleaning system and the second cleaning system.

Optionally, the fluid may be water.

According to another aspect of the present invention, there is provided an endoscope. The endoscope includes a first optical system to obtain an image of an observation object in predetermined magnifying power, a second optical system to obtain the image of the observation object in different magnifying power from the magnifying power of the first optical system, a first cleaning system to discharge fluid toward a first front end surface of the first optical system to clean the first front end surface, a second cleaning system to discharge the fluid toward a second front end surface of the second optical system to clean the second front end surface, and a switching system to switch flow of the fluid to be fed to one of the first cleaning system and the second cleaning system. The flow to be fed to the first cleaning system and the second cleaning system is stored in a single reservoir.

Optionally, the first cleaning system may be provided with a dischargeable portion, which is protruded forward from the front end surface of the first optical system when the fluid is discharged from the dischargeable portion and retracted rearward when the image of the observation object is obtained by the first optical system.

Optionally, the first optical system may be a confocal optical system, which is placed to be in direct contact with the observation object, capable of emitting illuminating light to the observation object and extracting light from the observation object, which is in a predetermined position with respect to the first optical system, and the first cleaning system may be capable of being advanced and retracted in a direction of an optical axis of the first optical system.

### BRIEF DESCRIPTION OF THE DRAWINGS

FIG. 1 is a diagram to schematically illustrate a cross-sectional side view of a confocal endomicroscope system according to a first embodiment of the present invention.

FIG. 2 is a front view of a distal end portion of a flexible insertion tube in the confocal endomicroscope system according to the first embodiment of the present invention.

FIGS. 3A-3C schematically illustrate switching unit of the confocal endomicroscope system according to the first embodiment of the present invention.

FIGS. 4A and 4B schematically illustrate a configuration of a nozzle portion in the confocal endomicroscope system according to the first embodiment of the present invention.

FIGS. 5A and 5B schematically illustrate a configuration of a nozzle portion in the confocal endomicroscope system according to a second embodiment of the present invention.

### DETAILED DESCRIPTION OF THE EMBODIMENT

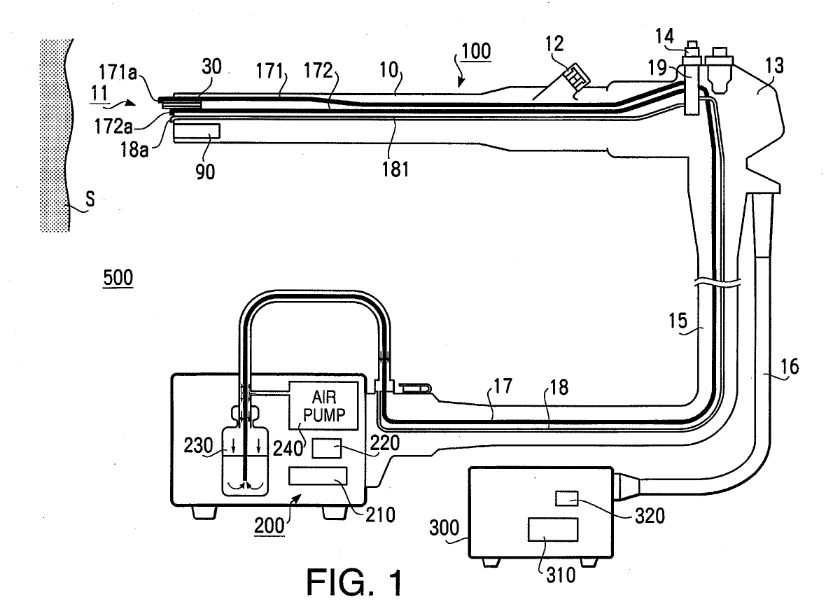

Hereinafter, referring to the accompanying drawings, an illustrative embodiment of the invention will be described. FIG. 1 is a diagram to schematically illustrate a cross-sectional side view of a confocal endomicroscope system 500 according to a first embodiment of the present invention. The confocal endomicroscope system 500 includes an electronic endoscope 100 to be inserted into a body cavity to capture images inside the body cavity, a first processor 200 and a second processor 300 to be respectively connected with the electronic endoscope 100. Each of the processors 200, 300 is connected with a monitor (not shown) to display the images output through the processors 200, 300.

The first processor 200, which is generally used for conventional observation, includes an image processing unit 210, a conventional light source unit 220, a reservoir 230, and an air pump 240. The second processor 300, which is used for confocal observation, includes an image processing unit 310 and a laser light source unit 320. In FIG. 1, other necessary components such as a light guiding member, including optical fibers to convey light and signal lines to convey electrical signals between a flexible insertion tube 10 and the image processing unit 210, the conventional light source unit 220, the image processing unit 310, the laser light unit 320 respectively, are omitted for simplicity in explanation.

The electronic endoscope 100 according to the present embodiment is provided with a conventional observation function, which enables capturing images of in vivo tissues in a body cavity with an image capturing optical system, e.g., a CCD (charge coupled device). Further, the electronic endoscope 100 is provided with a confocal observation function, which enables obtaining information of images of the body tissues in a body cavity with a confocal optical system 30. The electronic endoscope 100 includes the flexible insertion tube 10 to be inserted into the body cavity, a distal end portion 11 of the flexible insertion tube 10, a treatment tool inlet 12 through which a treatment tool such as forceps are inserted into the flexible insertion tube 10, an operation handle 13, which is held by an operator during an operation of the electronic endoscope 100, an operation unit 14 including various buttons and levers to control motions of the electronic endoscope 100, a first cable 15 and a second cable 16 to be respectively connected with the processors 200, 300.

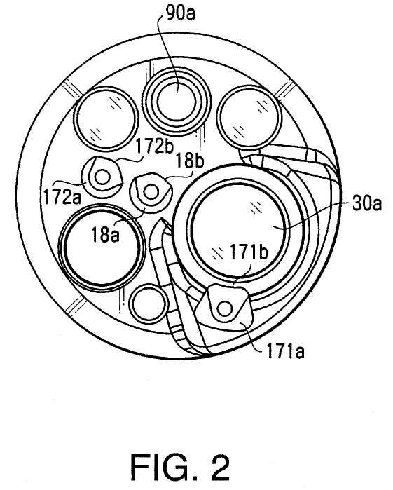

FIG. 2 is a front view of the distal end portion 11 of the flexible insertion tube 10 in the confocal endomicroscope system 500 according to the first embodiment of the present invention. The electronic endoscope 100 includes a liquid feed tube 17 and an air feed tube 18 to convey fluid to clean the front end surfaces of the confocal optical system 30 and an image capturing optical system 90. Liquid (e.g., water) for cleaning the front end surfaces of the confocal optical system 30 and the image capturing optical system 90 is stored in the reservoir 230 and conveyed through the liquid feed tube 17 to a switching unit 19 when the electronic endoscope 100 is connected with the first processor 200. The switching unit 19 is further connected with a first liquid tube 171 and a second liquid tube 172, and the water provided to the switching unit 19 through the liquid feed tube 17 is lead to one of the liquid tubes 171, 172.

The first liquid conducting tube 171 includes a nozzle portion 171a in vicinity to the distal end portion 11 of the flexible insertion tube 10. The water conducted through the first liquid conducting tube 171 is discharged from a discharge portion 171b of the nozzle portion 171a toward a front end surface 30a of the confocal optical system 30. The second liquid conducting tube 172 includes a nozzle portion 172a in vicinity to the distal end portion 11 of the flexible insertion tube 10. The water conducted through the second liquid conducting tube 172 is discharged from a discharge portion 172b of the nozzle portion 172a toward a front end surface 90a of the image capturing optical system 90. Thus, according to the present embodiment, the water stored in the single reservoir 230 can be discharged from either one of the two discharge portions 171b and 172b independently to clean corresponding one of the optical systems 30, 90.

Gas (e.g., air) is supplied by the air pump 240 and conveyed through the air feed tube 18 to the switching unit 19. The switching unit 19 is connected with an air conducting tube 181, and the air provided to the switching unit 19 through the air feed tube 18 is lead to the air conducting tube 181. The air conducting tube 181 includes a nozzle portion 18a in vicinity to the distal end portion 11 of the flexible insertion tube 10. The air conducted through the air conducting tube 181 is discharged from a discharge portion 18b of the nozzle portion 18a toward the front end surface 90a of the image capturing optical system 90.

The other front ends shown in FIG. 2 illustrate, for example, an illumination window, through which illuminating light is emitted, and a forceps channel, through which a treatment tool inserted through the treatment tool inlet 12 is exposed. However, detailed description of those front ends on the distal end portion 11 is herein omitted.

Next, a process to generate images for confocal observation within the confocal endomicroscope system 500, configured as above, to observe body tissue S will be described. During the confocal observation, the front end surface 30a of the confocal optical system 30 is required to be in direct contact with the body tissue S. Therefore, the electronic endoscope 100 according to the present embodiment is configured such that the front end surface 30a of the confocal optical system 30 protrudes further than the front end surface 90a of the image capturing optical system 90.

Laser beam, having a specific wavelength to act as excitation light and emitted from the laser light source unit 320, enters the confocal optical system 30 through a light guide member (not shown.) The confocal optical system 30 according to the present embodiment is configured such that a light emitting surface of the light guide member serves as a secondary point light source and as a confocal pinhole to specifically extract fluorescence, generated by the laser beam being emitted from the laser light source unit 320, from the body tissue S at the focused point of the irradiated light. The fluorescence from the body tissue S to be extracted thus may depend on positional relation with the confocal pinhole and the body tissue S. The confocal endomicroscope system 500 according to the present embodiment is configured such that the point light source can be advanced and retracted along the optical axis and shifted on a plane substantially perpendicular to the optical axis in a minute range. Thus, the laser beam emitted from the point light source can scan a surface of the body tissue substantially three-dimensionally.

Fluorescence generated from the body tissue S being illuminated by the laser beam is received at the light emitting surface to enter the light guide member and guided to the second processor 300. The fluorescence is therein separated from the light originating in the light source unit 320 by, for example, a fiber coupler (not shown) and received in the image processing unit 310. The image processing unit 310 processes the fluorescence being received to generate a point image to form a frame of still image. The confocal endomicroscope system 500 according to the present embodiment can display images formed as above according to a two-dimensional display method and a three-dimensional display method on the monitor. Further, cross-sectional images based on the three-dimensional image can be displayed. The image processing unit 310 processes the fluorescence according to the display method indicated by an operation from an operator through the operation unit 13.

A process for conventional observation in the confocal endomicroscope system 500 will be described. During the conventional observation, the body tissue S reflects light (e.g., white light) emitted from the conventional light source 220 in the first processor 200, and the reflection is received in an image capturing element (not shown) in the image capturing optical system 90. The image capturing element converts the reflection into image signals indicating the image of the body tissue S and passes the image signals to the image processing unit 210, in which an image is generated based on the image signals. The image is thereafter passed to the monitor to be displayed.

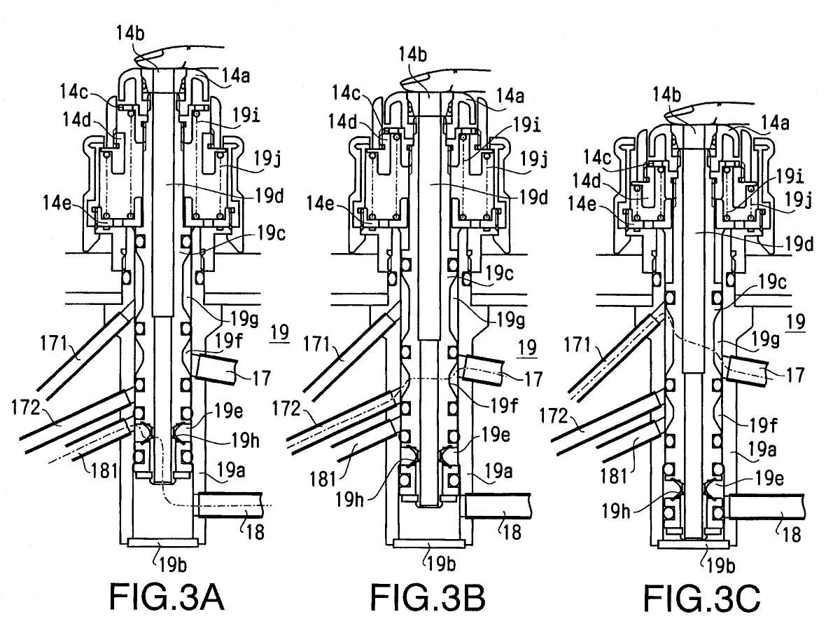

Referring to FIGS. 3A-3C, a configuration to convey the fluid in the confocal endomicroscope system 500 according to the present embodiment will be described. FIGS. 3A-3C schematically illustrate the switching unit 19 of the confocal endomicroscope system 500 according to the first embodiment of the present invention. Specifically, FIG. 3A illustrates the switching unit 19, through which air is conveyed from the air feed tube 18 to the air conducting tube 181. FIG. 3B illustrates the switching unit 19, through which water is conveyed from the liquid feed tube 17 to the second liquid tube 172. FIG. 3C illustrates the switching unit 19, through which water is conveyed from the liquid feed tube 17 to the first liquid tube 171.

As shown in FIGS. 3A-3C, the switching unit 19 is provided with a cylinder 19a. The cylinder 19a is sealed with a bottom cover 19b at a bottom thereof. The cylinder 19a is connected with the air feed tube 18, the air conducting tube 181, the second liquid tube 172, the liquid feed tube 17, and the first liquid tube 171, in an ascending order in FIGS. 3A-3C.

Inside the cylinder 19a, a piston 19c having a diameter which substantially fits an inner diameter of the cylinder 19a is provided. Thus, the piston 19c is capable of being advanced and retracted in an axial direction of the cylinder 19a (i.e., pressed down and pulled up vertically in FIGS. 3A-3C.) The piston 19c is provided with a button 14a, which is operated for controlling the fluid supply, at a top portion (i.e., remote from the bottom cover 19b) thereof. Thus, the switching unit is operated in association the operation to the button 14a. The piston 19c is formed to have a vent hole 19d, which is open at the top portion, at a position of the central axis of the piston 19c. In association with the vent hole 19d, the button 14a is formed to have a leakage hole 14b in a position corresponding to the vent hole 19d.

The piston 19c is further formed to have an air feed groove 19e, a second liquid feed groove 19f, and a first liquid feed groove 19g, in the ascending order in FIGS. 3A-3C, on the outer periphery. The air feed groove 19e is in communication with the vent hole 19d, however, a circular valve 19h is provided at a portion in which the vent hole 19d and the air feed groove 19e are interconnected. The valve 19h is resilient, formed to be circular, and has a cross-section of a V-shape. The piston 19c is further provided with O-rings to prevent leakage of the fluid in vicinity to each end of the grooves 19e, 19f, 19g.

The switching unit 19 includes first spring 19i and second spring 19j, which have different lengths from each other, to surround the piston 19c. The first spring 19i has a length which is greater than a length of the second spring 19j. The first spring 19i is provided between a first stopper 14c and a receiver base 14e, whilst the second spring 19j is provided between a second stopper 14d, which is in between the first stopper 14c and the receiver base 14e, and a receiver base 14e.

According to the effects of the first and the second springs 19i, 19j, the button 14a as well as the piston 19c can be pressed down in two steps from an initial position, in which the button 14a is not pressed at all. More specifically, it is noted that FIG. 3A shows the button 14a in the initial position. FIG. 3B shows the button 14a in a first pressed position, in which the first spring 19i is pressed until the first stopper 14c becomes in contact with the second stopper 14d. Starting from the first pressed position as shown in FIG. 3B, when the button 14a is pressed further down, both of the first spring 19i and the second spring 19j are pressed down until the second spring 19j is compressed to its maximum as shown in FIG. 3C (i.e., a second pressed position). FIG. 3C shows the button 14a in the second pressed position. In the present embodiment, the first spring 19i and the second spring 19j are configured to have different expanding forces. More specifically, the expanding force of the first spring 19i is less than the expanding force of the second spring 19j so that the operator can recognize the difference in pressing forces to press the button 14a down to the first pressed position and to the second pressed position, and easier operations of the button 14a can be achieved.

In the switching unit 19 as described above, when the air pump 240 (FIG. 1) is driven, air is conducted to the switching unit 19 through the air feed tube 18. In the initial position, as shown in FIG. 3A, the air feed tube 18 and the air conducting tube 181 can be communicated with each other through the vent hole 19d and the air feed groove 19e. However, in this state, if the operator does not seal the leakage hole 14b and leaves the leakage hole 14b open, the air is allowed to leak out of the switching unit 19 through the vent hole 19d and the leakage hole 14b without passing through the circular valve 19h, because the air pumped into the vent hole 19d needs to be compressed to a certain amount in order to pass through the circular valve 19h. Therefore, the air is prevented from flowing into the air feed groove 19e and the air conducting tube 181.

When the operator seals the leakage hole 14b of the button 14a at the initial position, as shown in FIG. 3A, the air pumped into the switching unit 19 through the air feed tube 18 is substantially compressed in the vent hole 19d to pass through the circular valve 19h to enter the air conducting tube 181, as indicated in the dash-and-dot line in FIG. 3A. The air is thus conducted through the air conducting tube 181, the nozzle portion 18a, the discharge portion 18b to the outside of the electronic endoscope 100. More specifically, the discharged air is directed toward the front end surface 90a of the image capturing optical system 90. Meanwhile, a surface level of the water in the reservoir 230 remains unaffected. Therefore, the obstacles on the front end surface 90a can be removed by the air pressure.

When the operator seals the leakage hole 14b and presses the button 14a downward, as shown in FIG. 3B, the liquid feed tube 17 and the second liquid tube 172 are communicated with each other through the second liquid feed groove 19f. In this state, the air feed tube 18 and the air conducting tube 181 are disconnected. Therefore, in this state, when the air pump 240 is driven, air is pumped into the reservoir 230 to press down the surface level of the water in the reservoir so that the water is conducted into the liquid feed tube 17. The water thus enters the second liquid tube 172 through the second liquid feed groove 19f, as indicated in the dash-and-dot line in FIG. 3B. The water is thus conducted through the second liquid tube 172, the nozzle portion 172a, the discharge portion 172b to the outside of the electronic endoscope 100. More specifically, the discharged water is directed toward the front end surface 90a of the image capturing optical system 90. Therefore, the obstacles on the front end surface 90a can be removed by the water pressure.

When the operator seals the leakage hole 14b and presses the button 14a downward further, as shown in FIG. 3C, the liquid feed tube 17 and the first liquid tube 171 are communicated with each other through the first liquid feed groove 19g. In this state, the air feed tube 18 and the air conducting tube 181 are disconnected. Therefore, in this state, when the air pump 240 is driven, air is pumped into the reservoir 230 to press down a surface level of the water in the reservoir so that the water is conducted into the liquid feed tube 17. The water thus enters the first liquid tube 171 through the first liquid feed groove 19g, as indicated in the dash-and-dot line in FIG. 3C. The water is thus conducted through the first liquid tube 171 to the nozzle portion 171a.

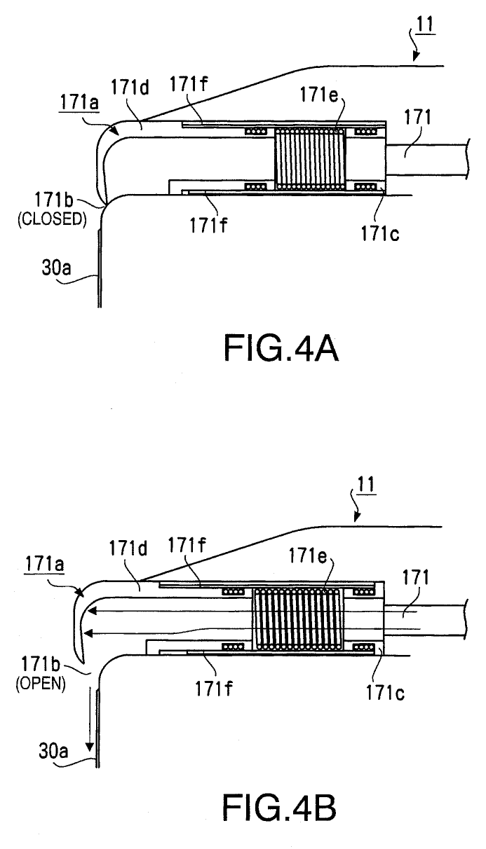

FIGS. 4A and 4B schematically illustrate a configuration of the nozzle portion 171a in the confocal endomicroscope system 500 according to the first embodiment of the present invention. FIG. 4A illustrates the nozzle portion 171a with no water being fed. FIG. 4B illustrates the nozzle portion 171a with water being fed.

The nozzle portion 171a includes a base 171c, a movable portion 171d, a helical extension spring 171e, and a guide 171f. The base 171c is fixed to the distal end portion 11 of the flexible insertion tube 10. The movable portion 171b is attached to the base 171c by the helical extension spring 171e and slidable along the guide 171f. The guide 171f is extended in a direction of the optical axis of the confocal optical system 30, which is perpendicular to the front end surface 30a of the confocal optical system 30. Therefore, the movable portion 171d is shifted along the guide 171f in the direction of the optical axis of the confocal optical system 30 as the helical extension spring 171e is expanded and compressed.

When water is not fed in the nozzle portion 171a, as shown in FIG. 4A, the helical extension spring 171e is compressed, therefore, the movable portion 171d is included in the distal end portion 11 of the flexible insertion tube 10. Thus, the discharge portion 171b is closed by an outer surface of the distal end portion 11. In this state, a front end surface of the nozzle portion 171a is aligned in a substantially same plane with the front end surface 30a of the confocal optical system 30. The nozzle portion 171a may be designed such that the front end surface of the nozzle portion 171a is retracted rearward from the plane on which the front end surface 30a is positioned as long as the discharge portion 171b is closed by the outer surface of the distal end portion 11.

When water is fed in the nozzle portion 171a (flow of the water is indicated by the arrows in FIG. 4B), the pressure of water presses an inner surface of the nozzle portion 171a. Therefore, as shown in FIG. 4B, the movable portion 171d is advanced along the guide 171f to protrude from the plane on which the front end surface 30a is positioned. As the movable portion 171d protrudes, the discharge portion 171b becomes open so that the water is discharged therethrough, and the front end surface 30a of the confocal optical system 30 can be cleaned. When the water stops, the movable portion 171d returns to the initial position (FIG. 4A) to be included in the distal end portion 11 due to a contracting effect of the helical extension spring 171e.

With the nozzle portion 171a configured as above, the front end surface of the nozzle portion 171a can be stored in the distal end portion 11 of the flexible insertion tube 10 when the observation is performed so that the nozzle portion 171a does not interfere with the front end surface 30a of the confocal optical system 30 being in contact with the body tissue S. The front end surface 30a of the confocal optical system 30 can be placed in direct contact with the body tissue S securely to obtain the confocal image.

During the cleaning operation, the water can be discharged through the discharge portion 171b with the nozzle portion 171a protruded from the front end surface 30a of the confocal optical system 30 so that the water flows down over the front end surface 30a to effectively and securely clean the front end surface 30a regardless of an amount of the front end surface 30a to be protruded with respect to the front end surface 90a of the image capturing optical system 90.

Although an example of carrying out the invention has been described above, the present invention is not limited to the above described embodiment. For example, a number of optical system, of which front end surface is cleaned, is not limited to two (i.e., the image capturing optical system 90 for conventional observation and the confocal optical system 30 in the above embodiment). When the endoscope system includes solely one optical system for observation, solely one set of water feeding tubes may be provided.

For another example, the nozzle portion to clean the surface of the optical system which can be retracted in the front end portion of the flexible insertion tube as described above may be applied to an image capturing optical system of an endoscope for conventional observation. In such a configuration, ghost reflection, in which illumination is reflected by a nozzle protruding outward from the front end surface of the optical system and which can deteriorate quality of images to be obtained, can be prevented.

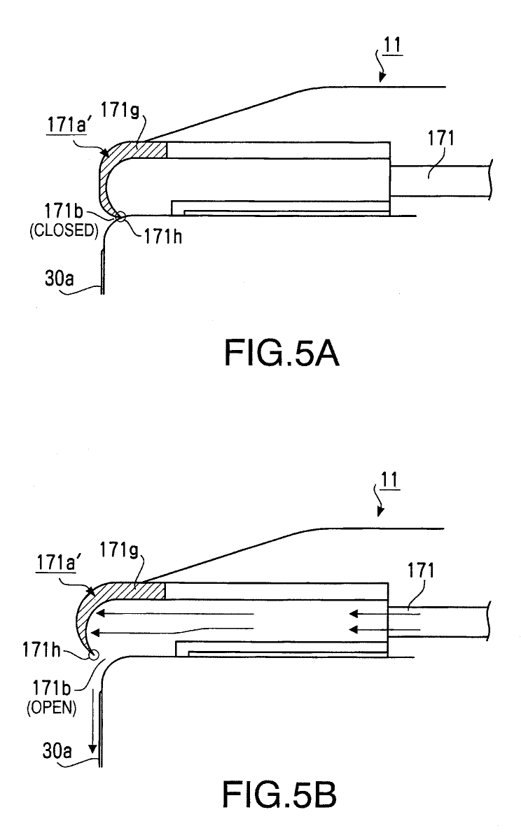

Further, the nozzle portion 171a may not be necessarily shifted along the guide 171f by the expanding force of the helical extension spring 171e as long as the nozzle portion 171a can be protruded outward from the front end surface 30a during the cleaning operation and retracted to be stored in the distal end portion 11 when the confocal optical system 30 is in direct contact with the body tissue S during the observation. An example of such a configuration is shown in FIGS. 5A and 5B as a second embodiment of the present invention.

FIGS. 5A and 5B schematically illustrate a configuration of a nozzle portion 171a′ according to the second embodiment of the present invention.

According to the second embodiment, the nozzle portion 171a′ includes a resilient tip end portion 171g made of, for example, rubber to form the discharge portion 171b. When water is not fed in the nozzle portion 171a′, as shown in FIG. 5A, the tip end portion 171g closes the opening, and the front end of the nozzle portion 171a′ remains within the plane on which the front end surface 30a is positioned.

When water is fed in the nozzle portion 171a′, as shown in FIG. 5B, the tip end portion 171g is resiliently deformed by pressure of the water. Thus, the discharge portion 171b becomes open so that the water is discharged therethrough toward the front end surface 30a of the confocal optical system 30. In this configuration, a tip 171h is formed to have a specific curvature to determine a size of the discharge portion 171b so that the water can be effectively discharged toward the front end surface 30a regardless of the deformed form of the tip end portion 171g. According to the above, the nozzle portion 171a′ can be provided in a more simple configuration than the configuration of the nozzle portion 171a in the first embodiment.

For another example of the present invention, the nozzle portions 171a, 171a′ may be aligned on (or retracted from) a same plane as the front end surface 30a when the front end surface 30a becomes in direct contact with the body tissue, i.e., during the confocal observation. In such a configuration, for example, the nozzle portion 171a shown in FIGS. 4A and 4B can be configured such that the movable portion 171d is protruded outward from the front end portion 30a for a predetermined amount in the initial position. The helical extension spring 171e is replaced with a helical compression spring. Thus, the nozzle portion 171a can be aligned on (or retracted from) the same plane as the front end surface 30a when the helical compression spring is compressed. Accordingly, when the front end surface 30a is in direct contact with the body tissue S, the movable portion 171d is pressed by the body tissue S so that the nozzle portion 171a can be stored in the front end portion of the flexible insertion tube 10.

Further, the liquid to clean the front end surface 30a of the confocal optical system 30 is not limited to water, but may be other cleaning liquid or gas such as air. Furthermore, the reservoir 230 and the air pump 240 may be equipped in the second processor 300 in stead of the first processor 300.

The present disclosure relates to the subject matter contained in Japanese Patent Application No. P2007-035051, filed on Feb. 15, 2007, which is expressly incorporated herein by reference in its entirety.

What is claimed is:

1. An endoscope system, comprising:
an endoscope having a first optical system to obtain an image of an observation object in predetermined magnifying power and a first cleaning system to discharge fluid toward a front end surface of the first optical system to clean the front end surface; and
a processor to process the image obtained through the endoscope, having a light source to illuminate the observation object and a reservoir to store the fluid for cleaning,
wherein a dischargeable portion of the first cleaning system is protruded forward from the front end surface of the first optical system when the fluid is discharged from the dischargeable portion and retracted rearward when the image of the observation object is obtained by the first optical system.

2. The endoscope system according to claim 1,
wherein the first optical system is a confocal optical system, which is placed to be in direct contact with the observation object, capable of emitting illuminating light to the observation object and extracting light from the observation object, which is in a predetermined position with respect to the first optical system.

3. The endoscope system according to claim 1,
wherein the dischargeable portion of the first cleaning system is retracted rearward to align on a plane on which the front end surface is positioned when the image of the observation object is obtained by the first optical system.

4. The endoscope system according to claim 1,
wherein the dischargeable portion of the first cleaning system is retracted rearward from a plane on which the front end surface is positioned when the image of the observation object is obtained by the first optical system.

5. The endoscope system according to claim 1,
wherein the dischargeable portion of the first cleaning system is protruded forward from the front end surface of the first optical system by pressure of the fluid to be discharged from the dischargeable portion.

6. The endoscope system according to claim 5,
wherein the dischargeable portion of the first cleaning system is formed by at least one deformable resilient member; and
wherein the dischargeable portion is protruded forward when the resilient member is deformed by the pressure of the fluid.

7. The endoscope system according to claim 6,
wherein the resilient member is provided in a position of the dischargeable portion to form a dischargeable opening when the fluid is fed in the dischargeable portion and the resilient member is deformed by the pressure of the fluid.

8. The endoscope system according to claim 7,
wherein the dischargeable opening is closed when the fluid is unfed in the dischargeable portion.

9. The endoscope system according to claim 5,
wherein the dischargeable portion of the first cleaning system is provided with a resilient spring; and
wherein the resilient spring is extended when the fluid is fed in the dischargeable opening so that the dischargeable portion is protruded forward from the front end surface of the first optical system by the pressure of the fluid and compressed when the fluid is unfed in the dischargeable portion so that the dischargeable portion is retracted.

10. The endoscope system according to claim 3,
wherein the dischargeable portion of the first cleaning system is provided with at least one compressible member; and
wherein the dischargeable portion of the first cleaning system is retracted rearward to align on the plane on which the front end surface of the first optical system is positioned when the front end surface is placed to be in direct contact with the observation object for confocal observation and the resilient member is compressed by pressure to place the front end surface of the first optical system in direct contact with the observation object.

11. The endoscope system according to claim 4,
wherein the dischargeable portion of the first cleaning system is provided with at least one compressible member; and
wherein the dischargeable portion of the first cleaning system is retracted rearward from the plane on which the front end surface is positioned when the front end surface is placed to be in direct contact with the observation object for confocal observation and the resilient member is compressed by pressure to place the front end surface of the first optical system in direct contact with the observation object.

12. The endoscope system according to claim 1, comprising:
a second optical system to obtain the image of the observation object in different magnifying power from the magnifying power of the first optical system; and
a second cleaning system to discharge the fluid toward a front end surface of the second optical system to clean the front end surface.

13. The endoscope system according to claim 12,
wherein the magnifying power of the first optical system is greater than the magnifying power of the second optical system; and
wherein the front end surface of the first optical system is protruded forward further than the front end surface of the second optical system.

14. The endoscope system according to claim 12,
wherein the first cleaning system and the second cleaning system are fed with the fluid being stored in the reservoir of the processor; and
wherein the endoscope system is provided with a switching system to switch flow of the fluid to be fed to one of the first cleaning system and the second cleaning system.

15. The endoscope system according to claim 1, wherein the fluid is water.

16. An endoscope comprising:
a first optical system to obtain an image of an observation object in predetermined magnifying power;
a second optical system to obtain the image of the observation object in different magnifying power from the magnifying power of the first optical system;
a first cleaning system to discharge fluid toward a first front end surface of the first optical system to clean the first front end surface;
a second cleaning system to discharge the fluid toward a second front end surface of the second optical system to clean the second front end surface; and
a switching system to switch flow of the fluid to be fed to one of the first cleaning system and the second cleaning system,
wherein the flow to be fed to the first cleaning system and the second cleaning system is stored in a single reservoir.

17. The endoscope according to claim 16,
wherein the first cleaning system is provided with a dischargeable portion, which is protruded forward from the front end surface of the first optical system when the fluid is discharged from the dischargeable portion and retracted rearward when the image of the observation object is obtained by the first optical system.

18. The endoscope system according to claim 17,
wherein the first optical system is a confocal optical system, which is placed to be in direct contact with the observation object, capable of emitting illuminating light to the observation object and extracting light from the observation object, which is in a predetermined position with respect to the first optical system; and
wherein the first cleaning system is capable of being advanced and retracted in a direction of an optical axis of the first optical system.
